(19) United States
(12) Patent Application Publication
(10) Pub. No.: US 2019/0231182 A1
(43) Pub. Date: Aug. 1, 2019

(54) ENDOSCOPE WITH OSCILLATION FOR SPECTRALLY-ENCODED ENDOSCOPY

(71) Applicant: Canon U.S.A., Inc., Melville, NY (US)

(72) Inventors: Jacob Schieffelin Brauer, Cambridge, MA (US); Zhuo Wang, Santa Clara, CA (US); Alex Altshuler, Cambridge, MA (US); Anderson Thi Mach, Cambridge, MA (US)

(21) Appl. No.: 15/886,254

(22) Filed: Feb. 1, 2018

(51) Int. Cl. A61B 1/07; A61B 1/00; A61B 5/00; G02B 23/26

(52) U.S. Cl. CPC A61B 1/07; A61B 1/00172; A61B 5/0075; A61B 1/0661; A61B 1/00009; A61B 1/00045; G02B 23/26

(57) ABSTRACT

Some systems comprise a light source; a spectrometer; an optical scope that has a longitudinal axis and that includes a rotatable optical assembly, which is coupled to the light source and to the spectrometer; and an oscillating-drive device that is coupled to the optical scope. Also, the oscillating-drive device and the optical scope are configured such that the oscillating-drive device can oscillate the rotatable optical assembly of the optical scope through greater than 90° of rotation around the longitudinal axis of the optical scope. And some systems comprise an optical scope that includes a rotatable optical assembly and a spooling mechanism. The rotatable optical assembly includes one or more light-guiding components. Furthermore, the spooling mechanism is configured to wind or unwind the one or more light-guiding components when the rotatable optical assembly rotates.

### BACKGROUND

This application generally concerns endoscopes.

An endoscope for spectrally-encoded endoscopy (SEE) is configured to scan a spectrally-encoded light across a sample. The reflected light from the sample is captured through one or more of the SEE's optical fibers, which transmit the reflected light to a spectrometer. Positional information from the scan is combined with spectral information from the spectrometer to create an image of the sample.

### SUMMARY

Some embodiments of a system comprise a light source, a spectrometer, an optical scope that has a longitudinal axis and that includes a rotatable optical assembly, and an oscillating-drive device that is coupled to the optical scope. The optical scope is coupled to the light source and to the spectrometer. Also, the oscillating-drive device and the optical scope are configured such that the oscillating-drive device can rotate the rotatable optical assembly of the optical scope through greater than 90° of rotation around the longitudinal axis of the optical scope.

Some embodiments of a system comprise an optical scope and an oscillating-drive device that is coupled to the optical scope. The optical scope includes a rotatable optical assembly and has a longitudinal axis. The oscillating-drive device and the optical scope are configured such that the oscillating-drive device can oscillate the rotatable optical assembly through more than 90° of rotation around the longitudinal axis of the optical scope.

Some embodiments of a system comprise an optical scope that includes a rotatable optical assembly and comprise a spooling mechanism. The optical assembly includes one or more light-guiding components. Also, the spooling mechanism is configured to wind and unwind the one or more light-guiding components as the rotatable optical assembly rotates.

### BRIEF DESCRIPTION OF THE DRAWINGS

FIG. 1A illustrates an example embodiment of an endoscopy system.

FIG. 1B illustrates a cross-sectional view of an example embodiment of an optical scope.

FIG. 1C illustrates a partially-cutaway view of the distal end of an example embodiment of an optical scope.

FIGS. 1D and 1E illustrate examples of scanning patterns.

FIG. 2 illustrates an example embodiment of an endoscopy system.

FIG. 3A illustrates an example embodiment of an endoscopy system.

FIG. 3B illustrates a perspective view of the example embodiment of an endoscopy system in FIG. 3A.

FIG. 4 illustrates an example embodiment of an endoscopy system.

FIG. 5 illustrates an example embodiment of an endoscopy system.

FIG. 6 illustrates an example embodiment of an endoscopy system.

FIG. 7 illustrates an example embodiment of an endoscopy system.

FIG. 8 illustrates an example embodiment of a spooling mechanism.

FIG. 9A illustrates an example embodiment of a spooling mechanism.

FIG. 9B illustrates a perspective view of the example embodiment of a spooling mechanism in FIG. 9A.

FIG. 10A illustrates an example embodiment of a spooling mechanism.

FIG. 10B illustrates a perspective view of the example embodiment of a spooling mechanism in FIG. 10A.

FIG. 11A illustrates an example embodiment of a spooling mechanism.

FIG. 11B illustrates a perspective view of a part of the example embodiment of a spooling mechanism in FIG. 11A.

FIG. 12A illustrates an example embodiment of an endoscopy system.

FIG. 12B illustrates a cross-sectional view of an example embodiment of a spooling mechanism taken from line A-A in FIG. 12A.

FIG. 13A illustrates an example embodiment of an endoscopy system.

FIG. 13B illustrates an example embodiment of an oscillating mechanism.

FIG. 13C illustrates an example embodiment of an oscillating mechanism.

FIG. 14A illustrates an example embodiment of an endoscopy system.

FIG. 14B illustrates an example embodiment of an oscillating mechanism.

FIG. 15A illustrates an example embodiment of an oscillating mechanism.

FIG. 15B illustrates an example embodiment of an oscillating mechanism.

FIG. 15C illustrates an example embodiment of an oscillating mechanism.

FIG. 15D illustrates an example embodiment of an oscillating mechanism.

FIG. 16 illustrates an example embodiment of an endoscopy system.

FIG. 17 includes graphs that show examples of the rotational positions of a rotatable optical assembly of an optical scope.

### DETAILED DESCRIPTION

The following paragraphs describe certain explanatory embodiments. Other embodiments may include alternatives, equivalents, and modifications. Additionally, the explanatory embodiments may include several novel features, and a particular feature may not be essential to some embodiments of the devices, systems, and methods that are described herein.

Some optical scopes (e.g., an endoscope) are configured to capture images from inside a subject, such as a human patient. An optical scope may include a sheath and may include a rotatable optical assembly. At its distal tip, the rotatable optical assembly may include a lens, a spacer, a grating, and a mirror. Also, the rotatable optical assembly may include a drive cable and one or more light-guiding components (e.g., optical fibers). The lens and the mirror focus a beam of light, collect the beam of light, and guide the beam of light. The sheath may include a lumen that encircles the rotatable optical assembly, and the sheath may also include a window on its distal end that keeps the rotatable optical assembly separate from a patient's tissue and fluids. The sheath may also have a working channel for irrigation, drug delivery, suction, culture, passing balloons or other instruments, and circulating fluid to clear the window. Also, one or more optical fibers in the optical scope can be used to navigate the optical scope to an object (e.g., organs, tissues), deliver light to the object, and detect light that is reflected by the object.

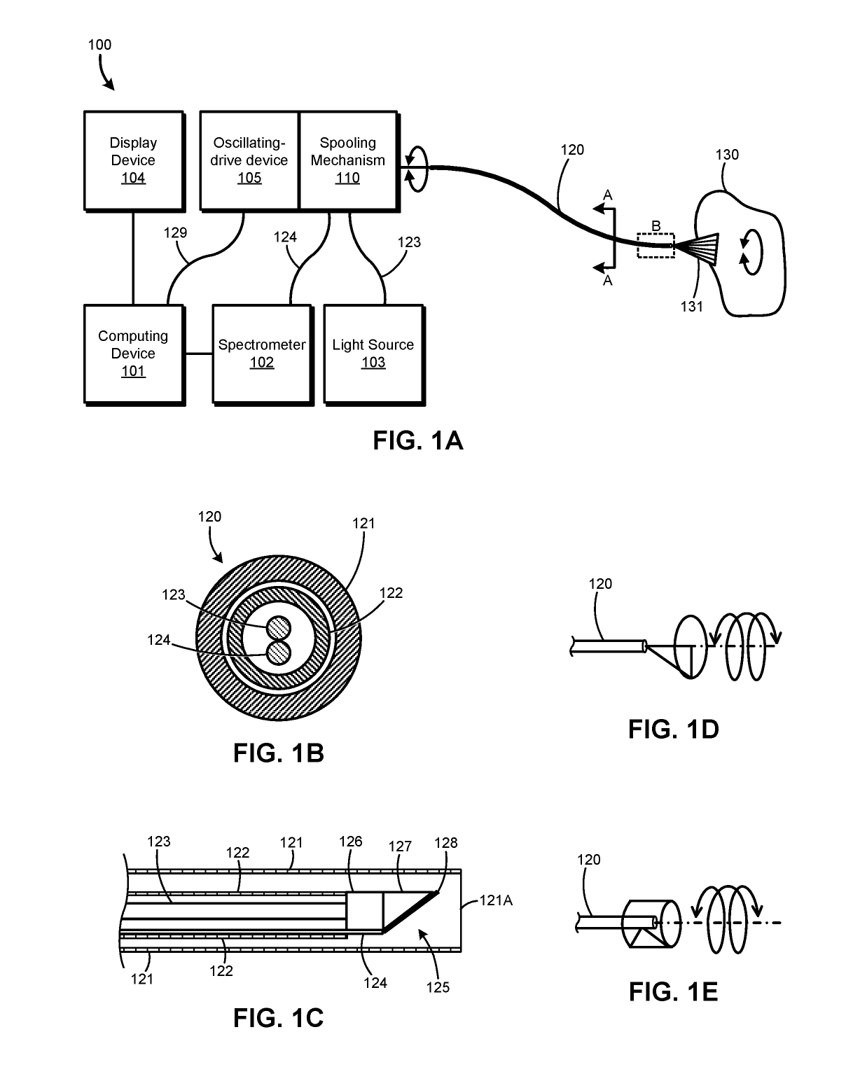

FIG. 1A illustrates an example embodiment of an endoscopy system 100. The endoscopy system 100 includes a computing device 101, a spectrometer 102, a light source 103, a display device 104, an oscillating-drive device 105, a spooling mechanism 110, and an optical scope 120. The computing device 101 communicates with (e.g., sends commands to) the oscillating-drive device 105 by means of a control cable 129.

The light source 103 is configured to emit light, and the light may be a broad-band light source with a short coherence length, for example a superluminescent light-emitting diode (SLED), a tunable light source, and a white-light source. The light may be a broadband light, and the light may have sufficient bandwidth to allow for spatial resolution along a spectrally-dispersed dimension. In some embodiments, the broadband light includes a blue band of light, a green band of light, or a red band of light. For example, the blue band may contain light in the range of 400-500 nm, the green band may contain light in the range of 500-600 nm, and the red band may contain light in the range of 600-800 nm. One or more light-guiding components (e.g., a first light-guiding component 123) carry the light from the light source 103 to a distal end of the optical scope 120. The optical scope 120 directs the light onto a sample 130. Additionally, the optical scope 120 receives light that has been reflected from the sample 130, and the optical scope 120 includes one or more light-guiding components (e.g., a second light-guiding component 124) that carry the received light back to the spectrometer 102. In some embodiments, one or both of the first light-guiding component 123 and the second light-guiding component 124 are optical fibers.

FIG. 1B illustrates a cross-sectional view of this example embodiment of the optical scope 120 taken from line A-A in FIG. 1A. Also, FIG. 1C illustrates a partially-cutaway view of the distal end (which is indicated by box B in FIG. 1A) of this example embodiment of the optical scope 120. This embodiment of the optical scope 120 includes a sheath 121 and a rotatable optical assembly 125. The sheath 121 includes a window 121A at its distal end. The rotatable optical assembly includes a drive cable 122, a first light-guiding component 123 (e.g., a single-mode fiber, a multi-mode fiber, a double-clad fiber), a second light-guiding component 124 (e.g., a single-mode fiber, a multi-mode fiber, a double-clad fiber), a light-focusing component 126, an angle-polished spacer 127, and a grating component 128.

The first light-guiding component 123 transmits light from the light source 103 to the distal end of the optical scope 120, where the light travels through the light-focusing component 126, the angle-polished spacer 127, and the grating component 128 and then scans the sample 130. The light-focusing component 126 may be, for example, a GRIN lens or a ball lens, and may be attached to the angle-polished spacer 127. The grating component 128 may be, for example, a binary grating, a blazed grating, or a holographic grating, and the grating component 128 may be fabricated by techniques such as dry-etching, wet-etching, nano-imprint, and soft lithography. Also, the grating component 128 may be formed directly on the angle-polished spacer 127. For example, an angle-polished spacer 127 with a grating component 128 may be fabricated by dicing and angle polishing an etched-glass grating.

The second light-guiding component 124 collects light that is reflected by the sample 130 and transmits the light to the spectrometer 102. In some embodiments, the second light-guiding component 124 is located in the sheath 121. In these embodiments, the second light-guiding component 124 is not part of the rotatable optical assembly 125.

The optical scope 120 follows a scanning pattern 131 as it directs light to, and receives reflected light from, the sample 130. FIGS. 1D and 1 E illustrate examples of scanning patterns. FIG. 1D shows the scanning pattern and the maximum field of view for an example embodiment of a forward-looking optical scope 120. The rotatable optical assembly 125 of the optical scope 120 can rotate more than 360° in either direction. FIG. 1E shows the scanning pattern and the maximum field of view for an example embodiment of a side-looking optical scope 120. The rotatable optical assembly 125 of the optical scope 120 can rotate more than 360° in either direction.

The oscillating-drive device 105 is configured to produce an oscillating torque that rotates the rotatable optical assembly 125 (e.g., by rotating the drive cable 122 of the optical scope 120). The spooling mechanism 110 allows the oscillating-drive device 105 to rotate the rotatable optical assembly 125 without breaking any of the light-guiding components that are in the rotatable optical assembly 125. Some embodiments of the oscillating-drive device 105 and the spooling mechanism 110 can be configured to rotate the rotatable optical assembly 125 through a range of ≤360° or through a range of >360°. For example, the rotational ranges can be from −720° to +720°, −360° to +360°, −180° to +180°, or −90° to +90°. Scans of ≤360° can be useful in some applications, where a view of ≤360° may be desirable or acceptable. Scans of >360° are relevant for many applications because scans of >360° reduce the percentage of time when the oscillating-drive device 105 and the rotatable optical assembly 125 are rapidly accelerating or rapidly decelerating. In some embodiments, the acceleration and deceleration zones are corrected by software or omitted from the creation of the image.

The spooling mechanism 110 dispenses and retracts the light-guiding components (e.g., the first light-guiding component 123 and the second light-guiding component 124) that rotate to prevent the light-guiding components from breaking because the light-guiding components rotate with the drive cable 122 but remain stationary at their proximal ends, for example where the proximal ends connect to the spectrometer 102 and the light source 103. Also, in some embodiments, the spooling mechanism 110 transmits the torque from the oscillating-drive device 105 to the drive cable 122 of the optical scope 120.

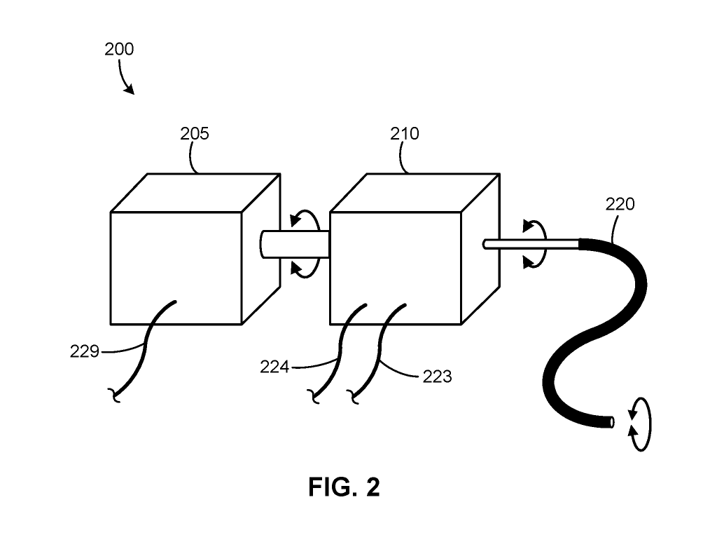

FIG. 2 illustrates an example embodiment of an endoscopy system 200. The system 200 includes an oscillating-drive device 205, a spooling mechanism 210, and an optical scope 220. A first light-guiding component 223 transmits light from a light source to the distal end of the optical scope 220, and a second light-guiding component 224 carries light that has been reflected from a sample back to a spectrometer. Also, a control cable 229 transmits control signals to the oscillating-drive device 205.

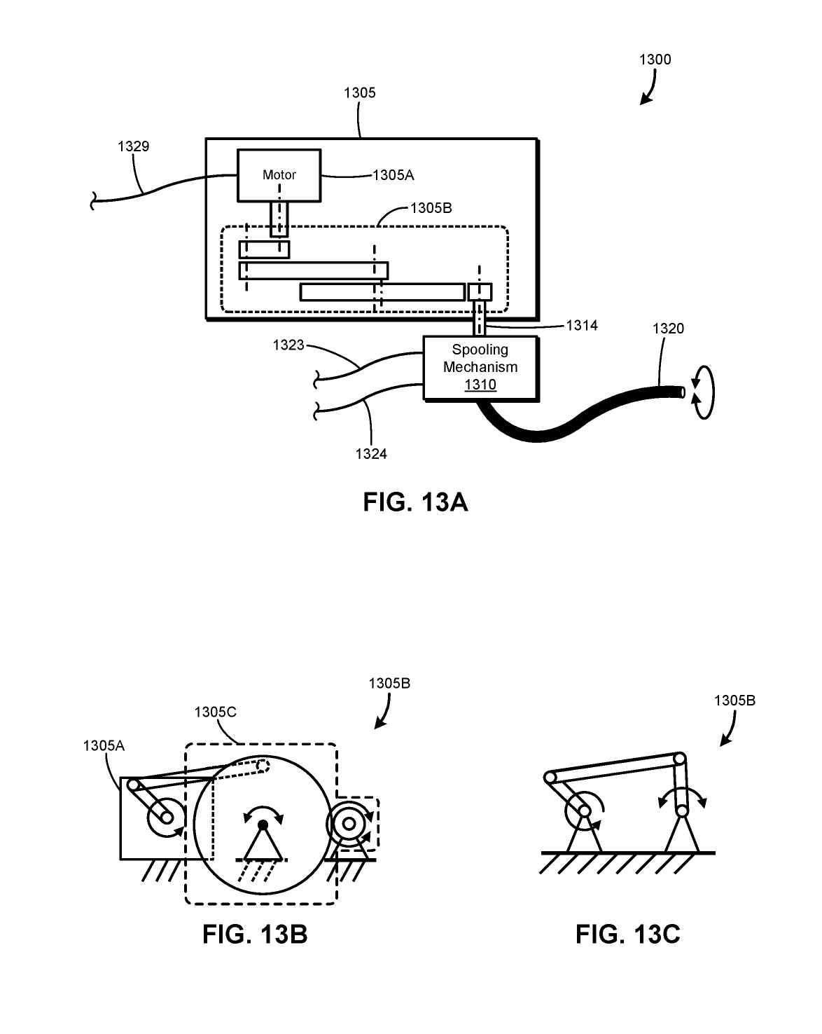

In this embodiment, the oscillating-drive device 205 is a motor that can be electronically controlled to oscillate back and forth (clockwise and counterclockwise). In some embodiments, the oscillating-drive device 205 accomplishes this by reversing the direction of the motor's rotation. And in some embodiments, the oscillating-drive device 205 includes a motor and another mechanism (e.g., the oscillating mechanisms shown in FIGS. 13B, 13C, 14B, 15A, 15B, 15C, and 15D) that converts a one-directional rotation of the motor into an oscillating torque. Also, the oscillating-drive device 205 can produce a torque that rotates a rotatable optical assembly of the optical scope 220 multiple times in each direction before reversing direction. The oscillating-drive device 205 may have a built-in position-sensing capability, or a position sensor may be coupled to the oscillating-drive device 205 or the spooling mechanism 210. And in some embodiments, a computing device (e.g., the computing device 101 in FIG. 1A) determines the rotational position of the rotatable optical assembly or the motor by analyzing the detection signal that is received from the second light-guiding component 224 of the optical scope 220.

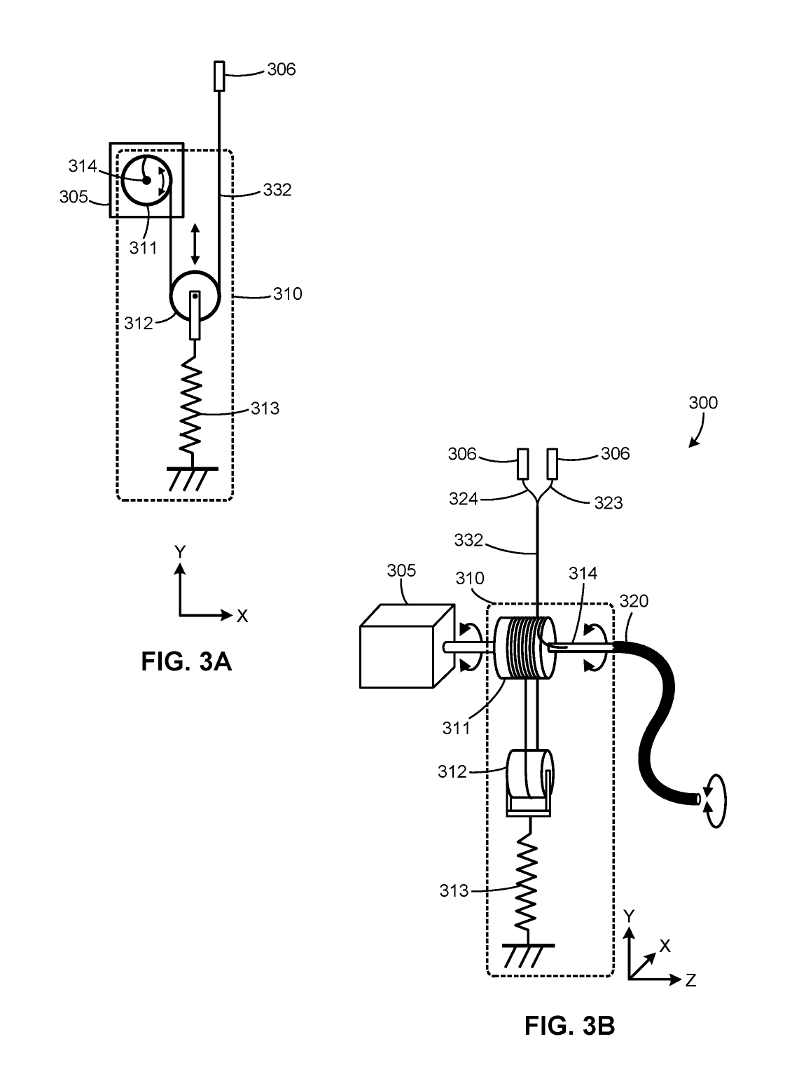

FIG. 3A illustrates an example embodiment of an endoscopy system, and FIG. 3B illustrates a perspective view of the example embodiment of an endoscopy system in FIG. 3A. The system 300 includes an oscillating-drive device 305, two stationary connectors 306, a spooling mechanism 310, and an optical scope 320. The spooling mechanism 310 includes a spool 311, a pulley 312, and a spring 313. The spooling mechanism 310 also has a coupling 314 to the oscillating-drive device 305 and to a drive cable of the optical scope 320. The oscillating-drive device 305 rotates the coupling 314, which rotates the spool 311 and a drive cable within the optical scope 320, as well as any components (e.g., a rotatable optical assembly) that are affixed to the drive cable.

In this embodiment, two light-guiding components 332 are wrapped around the spool 311 and run from the spool 311 through the pulley 312 to the two stationary connectors 306. The light-guiding components 332 may be jointly wound, jointly encased, or otherwise bound to each other. The light-guiding components 332 split into a first light-guiding component 323 and a second light-guiding component 324 near the two connectors 306.

The spring 313 allows the pulley 312 to move as the light-guiding components 332 are wound and unwound around the spool 311 as the spool 311 rotates, and the stiffness of the spring 313 is configured to prevent the light-guiding components 332 from breaking as the forces (e.g., tensile forces, shear forces) that are applied to the light-guiding components 332 increase and decrease during the winding and unwinding of the light-guiding components 332 around the spool 311.

In some embodiments, only the one or more light-guiding components that carry light from a light source to the distal end of the optical scope 320 rotate or twist relative to a sheath of the optical scope 320, and the one or more light-guiding components that carry light from the distal end of the optical scope 320 to a spectrometer remain stationary relative to the sheath of the optical scope 320.

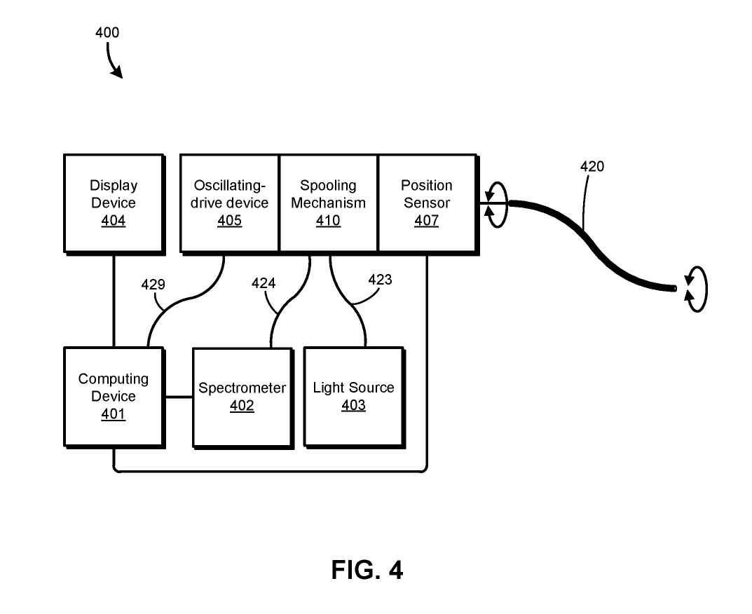

FIG. 4 illustrates an example embodiment of an endoscopy system. The endoscopy system 400 includes a computing device 401, a spectrometer 402, a light source 403, a display device 404, an oscillating-drive device 405, a position sensor 407, a spooling mechanism 410, and an optical scope 420. The position sensor 407 is coupled to the spooling mechanism 410. Also, the computing device 401 communicates with the oscillating-drive device 405 by means of a control cable 429.

One or more first light-guiding components 423 carry the emitted light from the light source 403 to a distal end of the optical scope 420. One or more second light-guiding components 424 carry received light back to the spectrometer 402.

In this embodiment, the position sensor 407 captures information about the rotational position of a part of the spooling mechanism (e.g., the spooling mechanism's spool, the spooling mechanism's coupling to a drive cable). Also, in some embodiments of the system 400, the position sensor 407 is coupled to the oscillating-drive device 405 and captures information about the rotational position of a part of the oscillating-drive device 405.

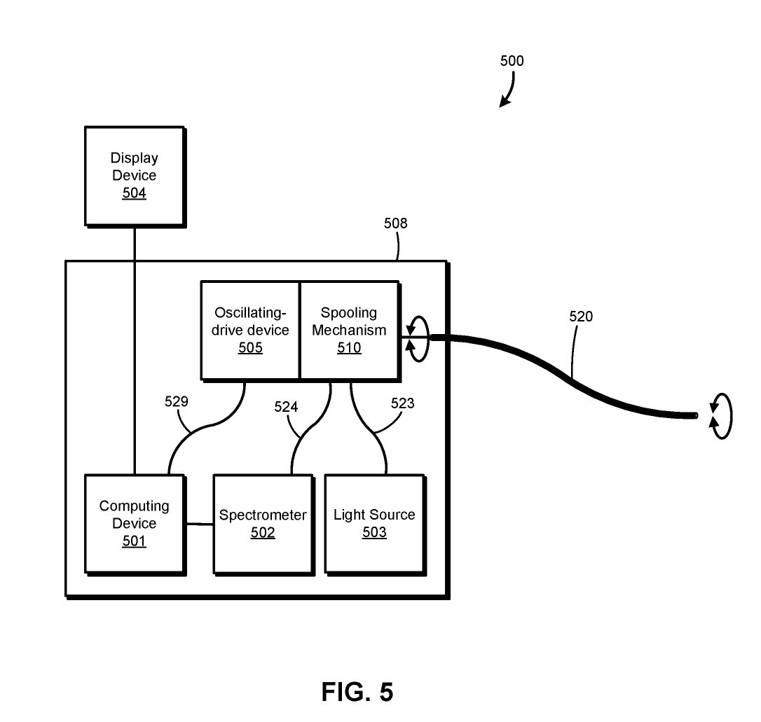

FIG. 5 illustrates an example embodiment of an endoscopy system. The endoscopy system 500 includes a computing device 501, a spectrometer 502, a light source 503, a display device 504, an oscillating-drive device 505, a spooling mechanism 510, and an optical scope 520. The computing device 501 communicates with the oscillating-drive device 505 by means of a control cable 529.

One or more first light-guiding components 523 carry the emitted light from the light source 503 to a distal end of the optical scope 520. And one or more second light-guiding components 524 carry received light back to the spectrometer 502.

In this embodiment, the computing device 501, the spectrometer 502, the light source 503, the display device 504, the oscillating-drive device 505, and the spooling mechanism 510 are members of a console 508. The console 508 may minimize the number of components that a user must handle when operating or transporting the system 500. The architecture of this system 500 may be particularly useful in applications where the console 508 must be covered with sterile drapes or in applications where sterility is less of a concern and it is acceptable to have the console 508 close to a patient. Keeping the console 508 close to the patient can decrease the length of the optical scope 520, and decreasing the length of the optical scope 520 may reduce the Non-Uniform Rotational Distortion (NURD). The display device 504 may be located away from the console 508, for example in a clinic where a physician has a wall-mounted display device 504 that can be connected to the console 508.

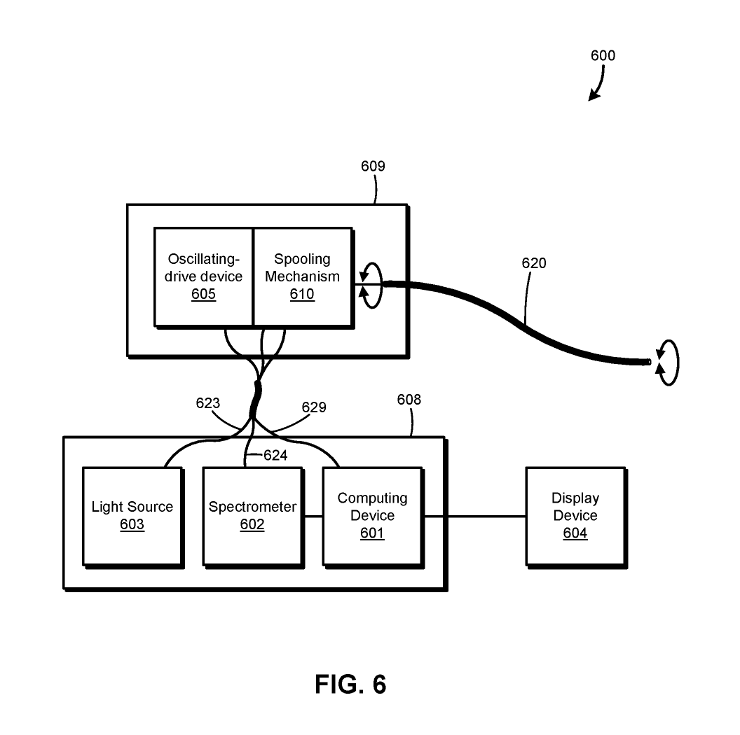

FIG. 6 illustrates an example embodiment of an endoscopy system. The endoscopy system 600 includes a computing device 601, a spectrometer 602, a light source 603, a display device 604, an oscillating-drive device 605, a spooling mechanism 610, and an optical scope 620. The computing device 601 communicates with the oscillating-drive device 605 by means of a control cable 629.

One or more first light-guiding components 623 carry the emitted light from the light source 603 to a distal end of the optical scope 620. Additionally, one or more second light-guiding components 624 carry received light back to the spectrometer 602.

In this embodiment, the computing device 601, the spectrometer 602, and the light source 603 are members of a console 608. Also, the oscillating-drive device 605 and the spooling mechanism 610 are members of a patient-interface unit 609. The patient-interface unit 609 allows a user to keep the console 608 outside of a patient's sterile field while also minimizing the length of the optical scope 620, which reduces NURD. And the patient-interface unit 609 can be bagged, encased, or otherwise covered for sterility.

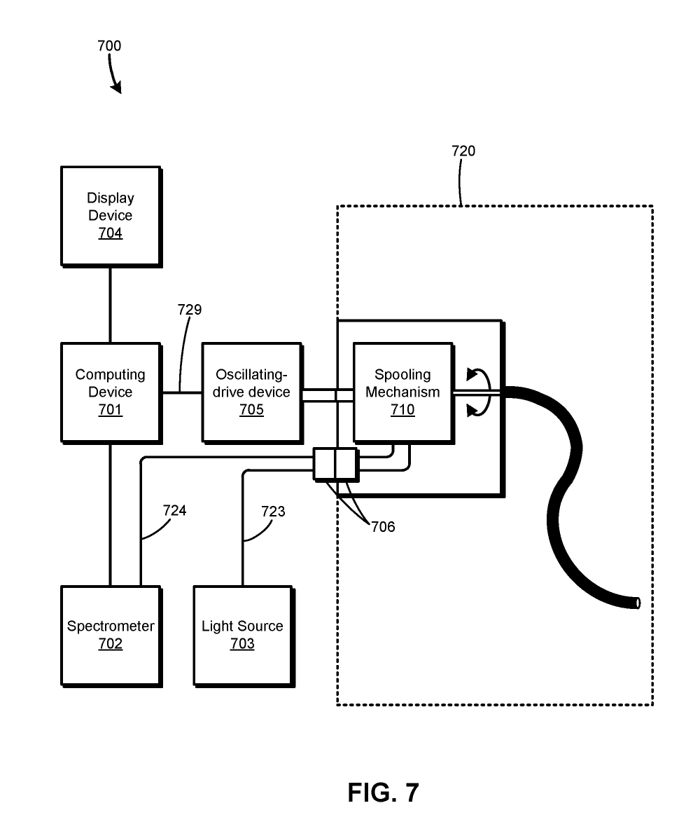

FIG. 7 illustrates an example embodiment of an endoscopy system. The endoscopy system 700 includes a computing device 701, a spectrometer 702, a light source 703, a display device 704, an oscillating-drive device 705, a spooling mechanism 710, and an optical scope 720. The computing device 701 communicates with the oscillating-drive device 705 by means of a control cable 729.

One or more first light-guiding components 723 carry the light from the light source 703 to a distal end of the optical scope 720. Additionally, one or more second light-guiding components 724 carry received light back to the spectrometer 702.

In this embodiment, the spooling mechanism 710 is a member of the optical scope 720. The optical scope 720 may be easily detached from the other members of the system 700 and be disposable. This embodiment may help to reduce fatigue in the spooling mechanism 710. A spooling mechanism 710 that is disposed of after one use does not need to survive for the same lifetime as the computing device 701, the spectrometer 702, the light source 703, the display device 704, and the oscillating-drive device 705.

The system 700 also includes connectors 706 that allow the first light-guiding component 723 and the second light-guiding component 724 to be split when the optical scope 720 is detached from the rest of the system 700.

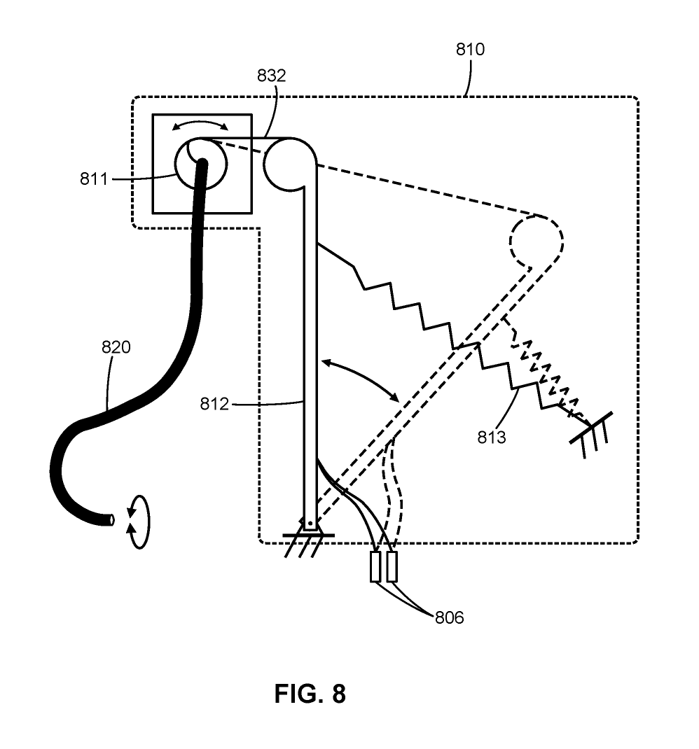

FIG. 8 illustrates an example embodiment of a spooling mechanism. The spooling mechanism 810 includes a spool 811, a flex arm 812, and a spring 813. The spool 811 is coupled to a drive cable of an optical scope 820. In this embodiment, two or more light-guiding components 832 are wrapped around the spool 811 and run from the spool 811 around the flex arm 812 to two connectors 806.

The flex arm 812 moves back and forth as the spool 811 winds and unwinds, taking up the slack in the light-guiding components 832 from the spool 811. Because of the length of the flex arm 812, the spool 811 can spin multiple times in either direction without causing much movement of the light-guiding components 832 at the connectors 806. In some embodiments, the flex arm 812 and the spring 813 are integrated, for example in embodiments in which the flex arm 812 includes a leaf spring.

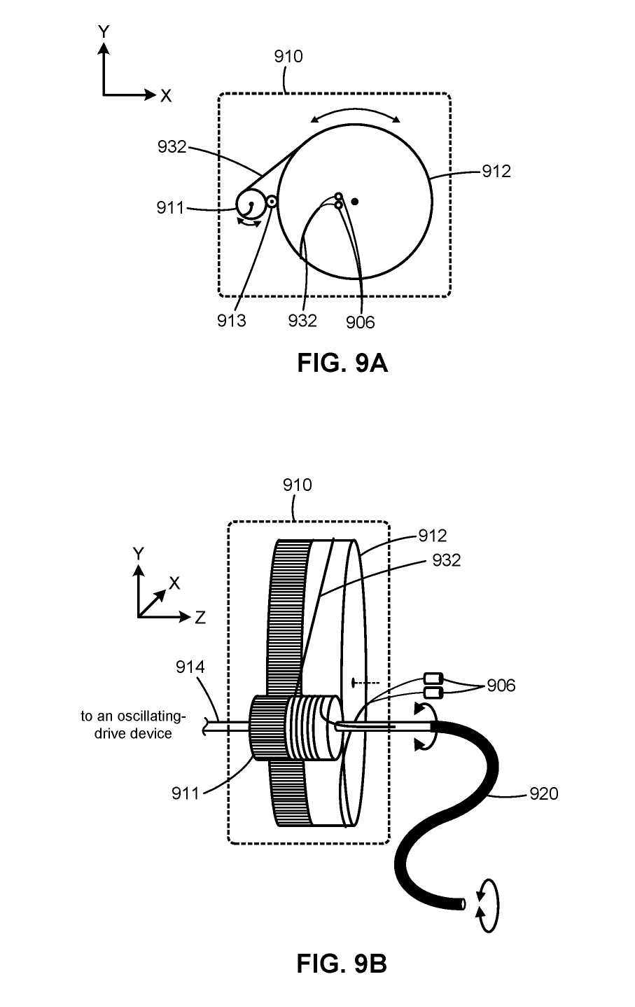

FIG. 9A illustrates an example embodiment of a spooling mechanism, and FIG. 9B illustrates a perspective view of the example embodiment of a spooling mechanism in FIG. 9A. The spooling mechanism 910 includes a small spool 911, a large spool 912, and an idler 913. The spooling mechanism 910 also has a coupling 914 to both an oscillating-drive device and a rotatable optical assembly of an optical scope 920. In this example embodiment, the coupling 914 passes through the small spool 911 and transfers a rotational force from the oscillating-drive device to the small spool 911 and to the rotatable optical assembly of the optical scope 920.

Also, two or more light-guiding components 932 are wrapped around the small spool 914 and the big spool 912, and the light-guiding components 932 run to two stationary connectors 906. The light-guiding components 932 split near the two connectors 906. Additionally, some embodiments include only one light-guiding component 932 and one connector 906.

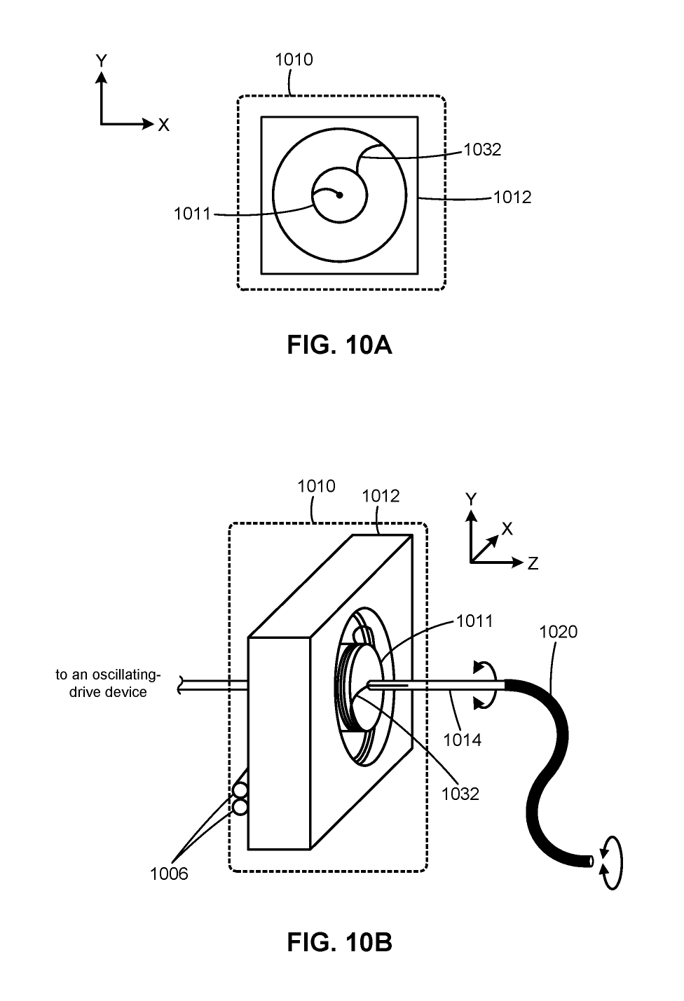

FIG. 10A illustrates an example embodiment of a spooling mechanism, and FIG. 10B illustrates a perspective view of the example embodiment of a spooling mechanism in FIG. 10A. The spooling mechanism 1010 includes an inner spool 1011 and an outer spool 1012. The spooling mechanism 1010 also has a coupling 1014 to an optical scope 1020 and an oscillating-drive device. In this example embodiment, the coupling 1014 passes through the inner spool 1011 and transfers a rotational force from oscillating-drive device to the inner spool 1011 and to a rotatable optical assembly of the optical scope 1020.

In this embodiment, two or more light-guiding components 1032 are wrapped around the inner spool 1011 and the outer spool 1012, and the light-guiding components 1032 extend to two connectors 1006, which may have fixed positions. The light-guiding components 1032 split near the two connectors 1006.

In this embodiment, the outer spool 1012 is stationary, and the inner spool 1011 rotates. As the inner spool 1011 rotates, the light-guiding components 1032 wind and unwind in the outer spool 1012.

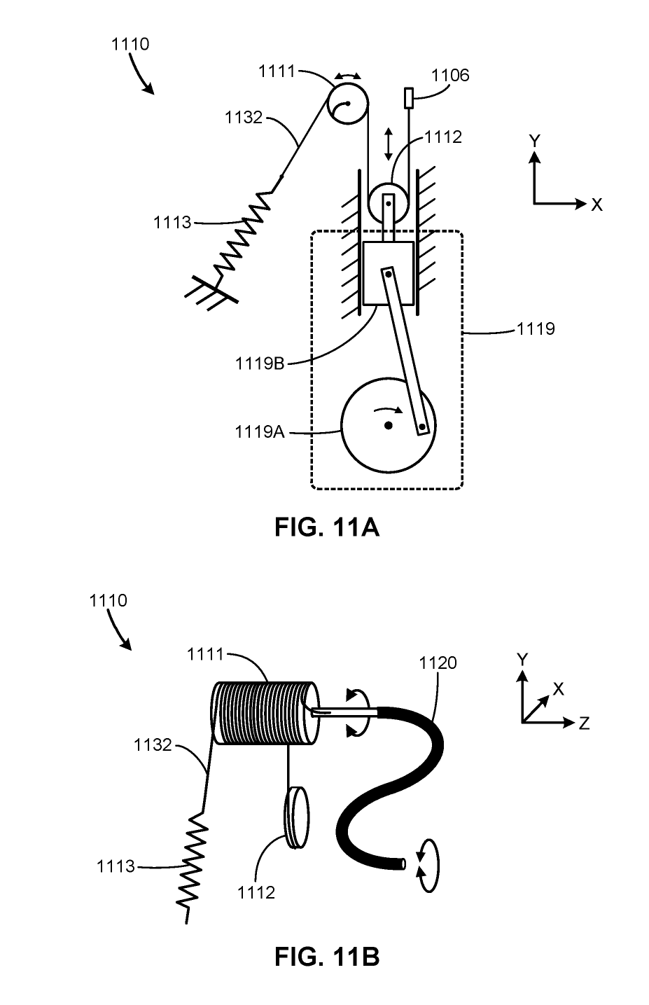

FIG. 11A illustrates an example embodiment of a spooling mechanism, and FIG. 11B illustrates a perspective view of part of the example embodiment of a spooling mechanism in FIG. 11A. The spooling mechanism 1110 includes an oscillating mechanism 1119, which is a slider crank 1119 in this example embodiment.

The spooling mechanism 1110 includes a spool 1111, a pulley 1112, a spring 1113, and the slider crank 1119. The spool 1111 is coupled to an optical scope 1120. In this embodiment, two or more light-guiding components 1132 are wrapped around the spool 1111 and run from the spool 1111 around the pulley 1112 to one or more connectors 1106. In some embodiments, the spring 1113 is a torsion spring that is mounted on the spool 1111.

The slider crank 1119 has a wheel 1119A that is connected to an oscillating-drive device or to drive device, which turns the wheel 1119A. In this embodiment, the wheel 1119A turns in only one direction. As the wheel 1119A rotates, it moves a slider 1119B up and down. As the slider 1119B moves up and down, it also moves the pulley 1112. As the pulley 1112 moves up and down, it pulls the light-guiding components 1132. The pulling forces of the pulley 1112 and of the spring 1113 cause the spool 1111 to rotate clockwise and counterclockwise. Also, in some embodiments, the two or more light-guiding components 1132 do not bear any tension: instead the tension is borne by a cable that runs alongside the light-guiding components 1132 or by a tube that encases the light-guiding components 1132.

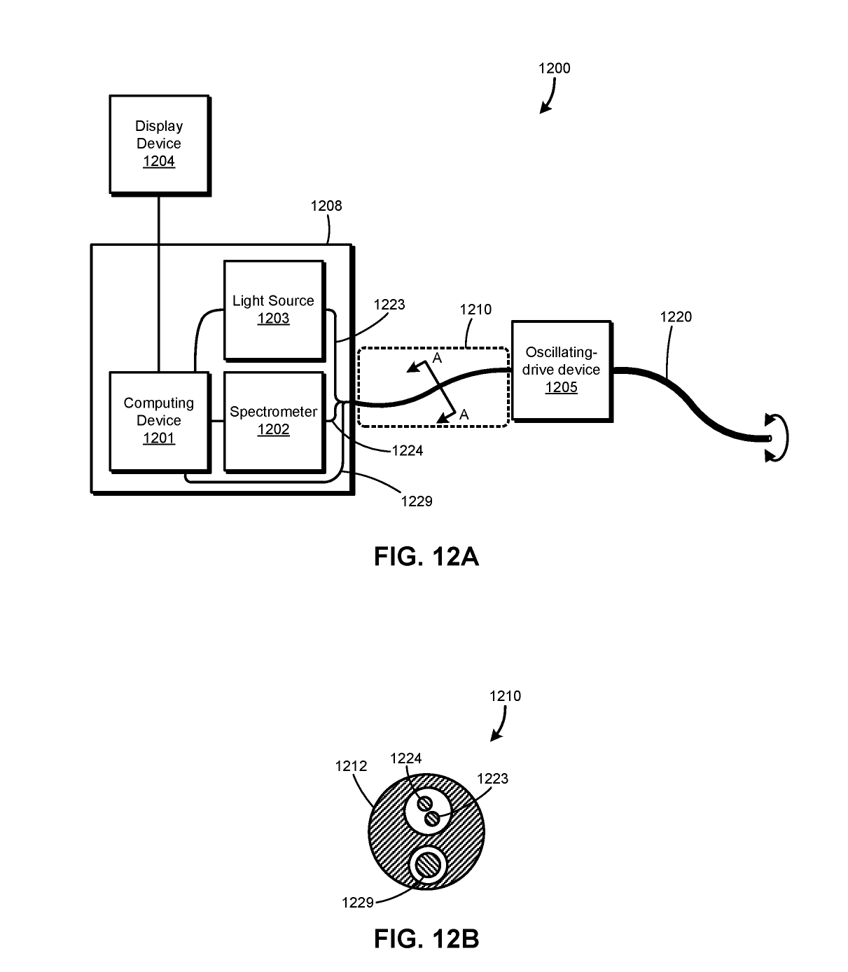

FIG. 12A illustrates an example embodiment of an endoscopy system. The endoscopy system 1200 includes a computing device 1201, a spectrometer 1202, a light source 1203, a display device 1204, an oscillating-drive device 1205, a spooling mechanism 1210, and an optical scope 1220. The computing device 1201 communicates with the oscillating-drive device 1205 by means of a control cable 1229.

Also, one or more first light-guiding components 1223 carry the emitted light from the light source 1203 to a distal end of the optical scope 1220, and one or more second light-guiding components 1224 carry received light back to the spectrometer 1202. In this embodiment, the computing device 1201, the spectrometer 1202, and the light source 1203 are members of a console 1208.

Additionally, in this embodiment, the spooling mechanism 1210 includes a protective tube 1212, and the spooling mechanism 1210 is positioned between the console 1208 and the oscillating-drive device 1205. The tube 1212 allows its internal light-guiding components to twist (e.g., into a double helix) and untwist. FIG. 12B illustrates a cross-sectional view of an example embodiment of the spooling mechanism 1210 taken from line A-A in FIG. 12A. The protective tube 1212 has a longitudinally-extending cavity that gives the first light-guiding component 1223 and the second light-guiding component 1224 enough space to twist and untwist. The protective tube 1212 also encloses the control cable 1229. In some embodiments, the protective tube 1212 is more than a meter long. Furthermore, in some embodiments, the protective tube 1212 is housed in the console 1208 or in a patient-interface unit.

FIG. 13A illustrates an example embodiment of an endoscopy system. The system 1300 includes an oscillating-drive device 1305, a spooling mechanism 1310, and an optical scope 1320. The oscillating-drive device 1305 transmits a rotational force to the spooling mechanism 1310 by means of a coupling 1314, which is a shaft in this example. A first light-guiding component 1323 transmits light from a light source to the distal end of the optical scope 1320, and a second light-guiding component 1324 carries light that has been reflected from a sample back to a spectrometer. Also, a control cable 1329 transmits signals (e.g., commands) from a computing device to the oscillating-drive device 1305.

In this example embodiment, the oscillating-drive device 1305 includes a motor 1305A and a four-bar linkage 1305B. The motor 1305A and the four-bar linkage 1305B generate the oscillating rotation. The motor 1305A is configured to spin continuously in one direction and power the four-bar-linkage 13058. The four-bar linkage 13058 converts the one-directional rotation of the motor 1305A into an oscillating rotation. The oscillating rotation of the four-bar linkage 13058 can be directly connected to a rotatable optical assembly of the scope 1320 for a more-limited angle of rotation, or it can be coupled to a transmission to increase the angle of rotation. In some embodiments, the four-bar linkage 1305B includes a transmission 1305C, for example a transmission 1305C that includes gears or a belt drive. Additionally, some embodiments of the four-bar linkage 1305B do not include a transmission, for example the embodiment of the four-bar linkage 1305B that is shown in FIG. 13C.

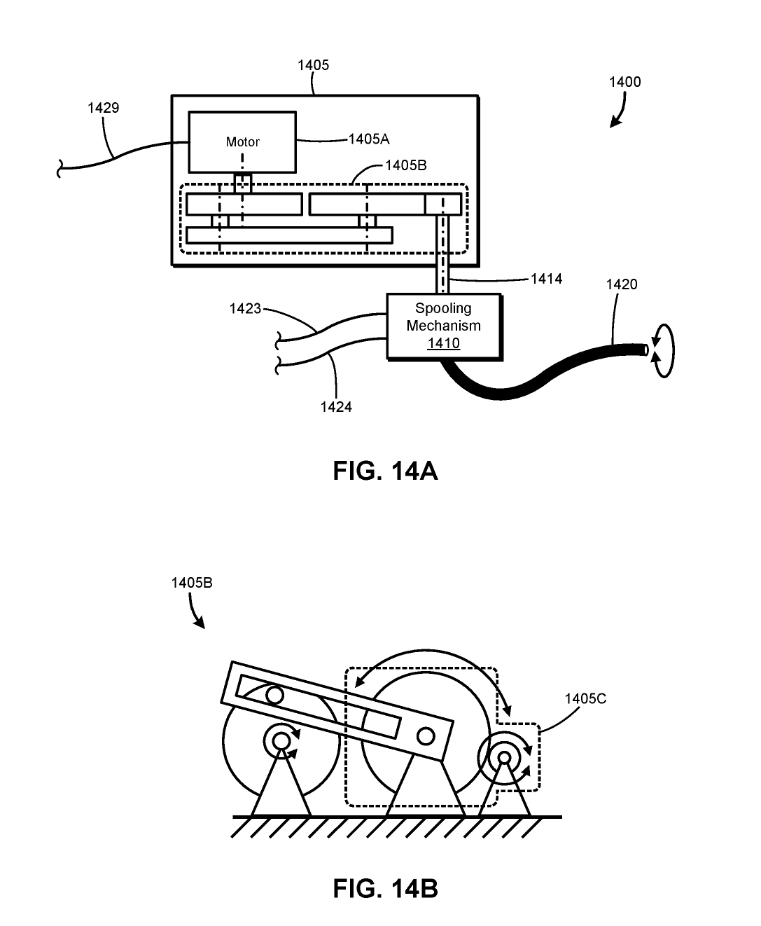

FIG. 14A illustrates an example embodiment of an endoscopy system. The system 1400 includes an oscillating-drive device 1405, a spooling mechanism 1410, and an optical scope 1420. The oscillating-drive device 1405 transmits a rotational force to the spooling mechanism 1410 by means of a coupling 1414, which is a shaft in this example. A first light-guiding component 1423 transmits light from a light source to the distal end of the optical scope 1420, and a second light-guiding component 1424 carries light that from the distal end to a spectrometer. Also, a control cable 1429 transmits signals (e.g., commands) to the oscillating-drive device 1405.

In this example embodiment, the oscillating-drive device 1405 includes a motor 1405A and a quick-return crank mechanism 1405B. This embodiment of the oscillating-drive device 1405 scans faster in one direction than the other, which may produce a saw-tooth scanning pattern when the optical scope 1420 is used for imaging. The oscillating rotation of the quick-return crank mechanism 14058 can be directly connected to the scope 1420 for a more-limited angle of rotation, or the quick-return crank mechanism 1405B can include a transmission 1405C that increases the angle of rotation. In some embodiments, the transmission 1405C includes gears or a belt drive.

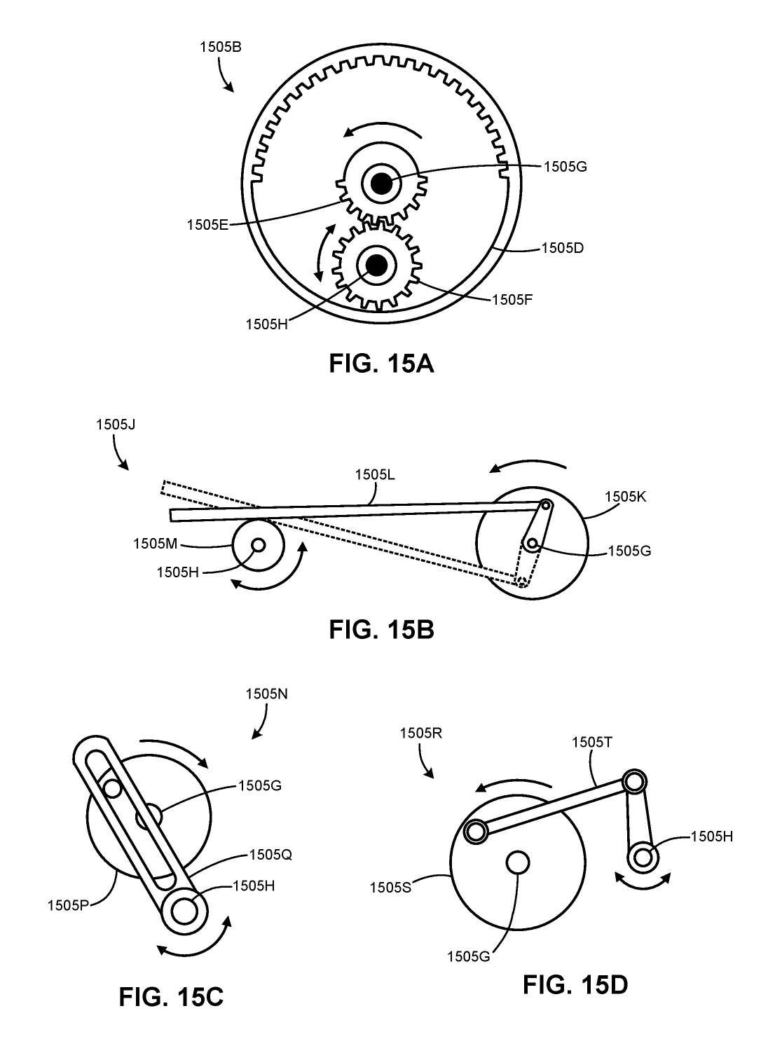

FIG. 15A illustrates an example embodiment of an oscillating mechanism, which is a quick-return crank mechanism in this example. The quick-return crank mechanism 1505B includes a first cogwheel 1505D, a second cogwheel 1505E, and a third cogwheel 1505F. The first cogwheel 1505D and the second cogwheel 1505E are fixed relative to each other. A first shaft 1505G extends through, and is connected to, the first cogwheel 1505D and the second cogwheel 1505E. Also, the first shaft 1505G is configured to be coupled to a motor that turns the first shaft 1505G, the first cogwheel 1505D, and the second cogwheel 1505 E in one direction. A second shaft 1505H extends through, and is connected to, the third cogwheel 1505F, and the second shaft 1505H is configured to be coupled to an optical scope's rotatable optical assembly (e.g., to a drive cable) or to a spooling mechanism. This quick-return crank mechanism 1505B produces a slow forward motion and a quick reverse motion. Also, some embodiments of the quick-return crank mechanism 1505B include friction wheels instead of cogwheels.

FIG. 15B illustrates an example embodiment of an oscillating mechanism. The oscillating mechanism 1505J includes a crank 1505K, an oscillating rod 1505L, and an output wheel 1505M. A first shaft 1505G extends through, and is connected to, the crank 1505K, and the first shaft 1505G is configured to be coupled to a motor that turns the first shaft 1505G and the crank 1505K in one direction. A second shaft 1505H extends through, and is connected to, the output wheel 1505M, and the second shaft 1505H is configured to be coupled to an optical scope's rotatable optical assembly (e.g., to a drive cable) or to a spooling mechanism. The oscillating mechanism 1505J converts the circular motion of the crank 1505K into the reciprocating motion of the output wheel 1505M and the second shaft 1505H.

FIG. 15C illustrates an example embodiment of an oscillating mechanism. The oscillating mechanism 1505N includes a rotating disk 1505P and a slotted arm 1505Q. A first shaft 1505G extends through, and is connected to, the rotating disk 1505P, and the first shaft 1505G is configured to be coupled to a motor that turns the first shaft 1505G and the rotating disk 1505P in one direction. A second shaft 1505H extends through, and is connected to, an end of the slotted arm 1505Q, and the second shaft 1505H is configured to be coupled to an optical scope's rotatable optical assembly (e.g., to a drive cable) or to a spooling mechanism. The oscillating mechanism 1505N converts the one-directional circular motion of the rotating disk 1505P into the alternating rotational motion of the slotted arm 1505Q and the second shaft 1505H.

FIG. 15D illustrates an example embodiment of an oscillating mechanism. The oscillating mechanism 1505R includes a rotating disk 1505S and a connecting rod 1505T. A first shaft 1505G extends through, and is connected to, the rotating disk 1505S, and the first shaft 1505G is configured to be coupled to a motor that turns the first shaft 1505G and the rotating disk 1505S in one direction. A second shaft 1505H extends through, and is connected to, an end of the connecting rod 1505T, and the second shaft 1505H is configured to be coupled to an optical scope's rotatable optical assembly (e.g., to a drive cable) or to a spooling mechanism. The oscillating mechanism 1505R converts the one-directional circular motion of the rotating disk 1505S into the alternating rotational motion of the second shaft 1505H.

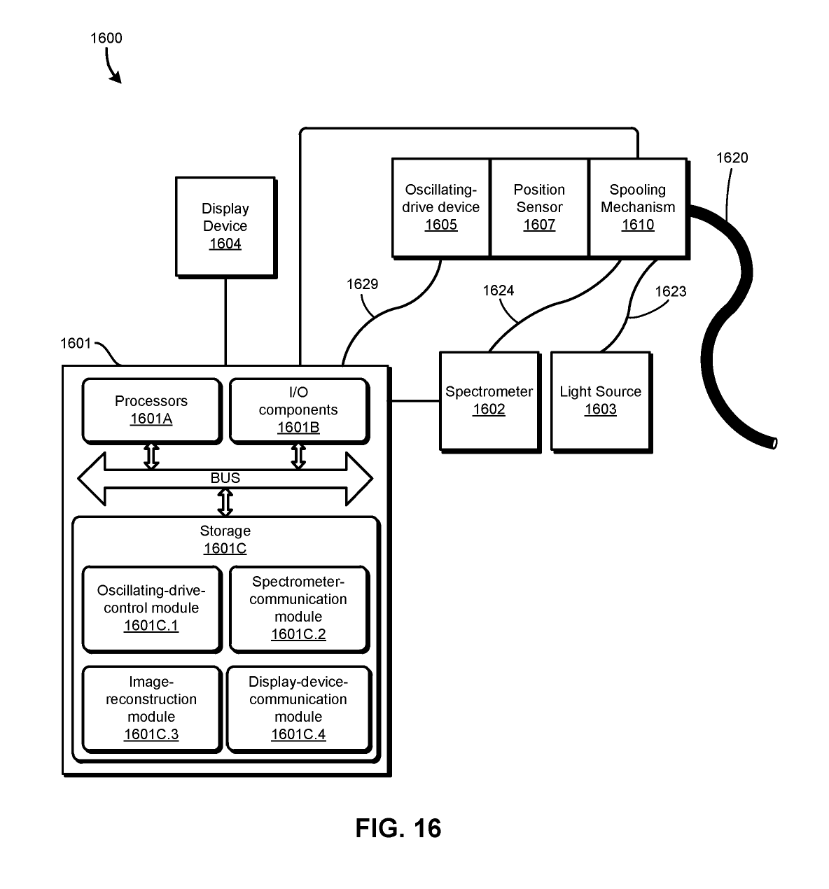

FIG. 16 illustrates an example embodiment of an endoscopy system. The endoscopy system 1600 includes a computing device 1601, a spectrometer 1602, a light source 1603, a display device 1604, an oscillating-drive device 1605, a position sensor 1607, a spooling mechanism 1610, and an optical scope 1620. The computing device 1601 communicates with the oscillating-drive device 1605 by means of a control cable 1629.

One or more first light-guiding components 1623 carry the emitted light from the light source 1603 to a distal end of the optical scope 1620. Additionally, one or more second light-guiding components 1624 carry received light from the distal end back to the spectrometer 1602.

The computing device 1601 is a specially-configured computing device that includes one or more processors 1601A, one or more I/O components 1601B, and storage 1601C. Additionally, the hardware components of the computing device 1601 communicate by means of one or more buses or other electrical connections. Examples of buses include a universal serial bus (USB), an IEEE 1394 bus, a PCI bus, an Accelerated Graphics Port (AGP) bus, a Serial AT Attachment (SATA) bus, and a Small Computer System Interface (SCSI) bus.

The one or more processors 1601A include one or more central processing units (CPUs), which include microprocessors (e.g., a single core microprocessor, a multi-core microprocessor); one or more graphics processing units (GPUs); one or more application-specific integrated circuits (ASICs); one or more field-programmable-gate arrays (FPGAs); one or more digital signal processors (DSPs); or other electronic circuitry (e.g., other integrated circuits).

The I/O components 1601B include communication components (e.g., a GPU, a network-interface controller) that communicate with input and output devices. Examples of input and output devices include a keyboard, a display device, a mouse, a printing device, a touch screen, a light pen, an optical-storage device, a scanner, a microphone, a drive, a controller (e.g., a joystick, a control pad), and a network.

The storage 1601C includes one or more computer-readable storage media. As used herein, a computer-readable storage medium, in contrast to a mere transitory, propagating signal per se, is a computer-readable media that includes an article of manufacture, for example a magnetic disk (e.g., a floppy disk, a hard disk), an optical disc (e.g., a CD, a DVD, a Blu-ray), a magneto-optical disk, magnetic tape, and semiconductor memory (e.g., a non-volatile memory card, flash memory, a solid-state drive, SRAM, DRAM, EPROM, EEPROM). Also, as used herein, a transitory computer-readable medium is a mere transitory, propagating signal per se, and a non-transitory computer-readable medium is any computer-readable medium that is not merely a transitory, propagating signal per se. The storage 1601C, which may include both ROM and RAM, can store computer-readable data or computer-executable instructions.

The computing device 1601 also includes an oscillating-drive-control module 1601C.1, a spectrometer-communication module 1601C.2, an image-reconstruction module 1601C.3, and a display-device-communication module 1601C.4. A module includes logic, computer-readable data, or computer-executable instructions, and may be implemented in software (e.g., Assembly, C, C++, C#, Java, BASIC, Perl, Visual Basic), hardware (e.g., customized circuitry), or a combination of software and hardware. In some embodiments, the computing device 1601 includes additional or fewer modules, the modules are combined into fewer modules, or the modules are divided into more modules. When the modules are implemented in software, the software can be stored in the storage 1601C.

The oscillating-drive-control module 1601C.1 includes instructions that, when executed, or circuits that, when activated, cause the computing device 1601 to generate signals (e.g., commands) that control the oscillating-drive device 1605. Also, the oscillating-drive-control module 1601C.1 may use positional information from the position sensor 1607 to generate the signals.

The spectrometer-communication module 1601C.2 includes instructions that, when executed, or circuits that, when activated, cause the computing device 1601 to obtain spectrometry data from the spectrometer 1602.

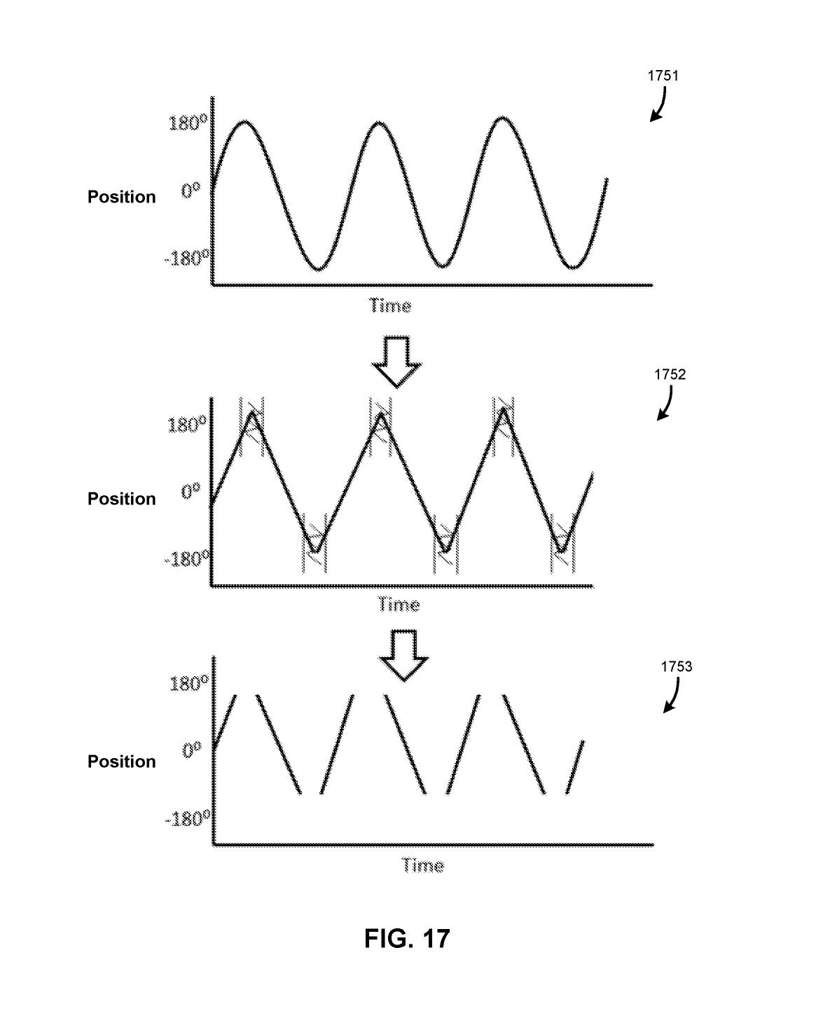

The image-reconstruction module 1601C.3 includes instructions that, when executed, or circuits that, when activated, cause the computing device 1601 to generate an image of a scanned object based on obtained spectrometry data and, in some embodiments, on positional information from the position sensor 1607. Furthermore, some embodiments of the image-reconstruction module 1601C.3 cause the computing device 1601 to determine the rotational position of the rotatable optical assembly of the optical prove 1620 based on obtained spectrometry data. Additionally, some embodiments of the image-reconstruction module 1601C.3 cause the computing device 1601 to linearize any non-linear movements of the optical scope 1620 or to delete portions of the spectrometry data that were captured while the optical scope 1620 was rapidly accelerating or decelerating, for example as shown in FIG. 17.

FIG. 17 includes graphs that show examples of the rotational positions of a rotatable optical assembly of the optical scope 1620. FIG. 17 shows the rotational position of a rotatable optical assembly of an optical scope on the y axis and shows time on the x axis. The first graph 1751 shows the rotational positions of the rotatable optical assembly of the optical scope 1620 as described by the positional information that was received from the position sensor 1607. The second graph 1752 shows the rotational positions of the rotatable optical assembly of the optical scope 1620 after the movements have been linearized. Additionally, the second graph 1752 indicates the areas where the rotatable optical assembly was rapidly accelerating or decelerating. The third graph 1753 shows the rotational positions of the optical scope 1620 after the areas where the rotatable optical assembly was rapidly accelerating or decelerating have been removed. Thus, the remaining positional information in the third graph 1753 has been linearized and does not include the areas where the optical scope 1620 was rapidly accelerating or decelerating. When reconstructing an image, some embodiments of the image-reconstruction module 1601C.3 use only the positional information in the second graph 1752 or in the third graph 1753 and the spectrometry data that was captured while the rotatable optical assembly was in the indicated positions.

Also, the scopes or sizes of the areas of rapid acceleration or deceleration, as distinguished from the areas of normal acceleration or deceleration, may be determined by evaluating an image constructed after removing the areas of rapid acceleration or deceleration. If the image is sufficiently clear, then the areas of rapid acceleration or deceleration were adequately removed. If the image is not sufficiently clear, then larger areas may be used for the areas of rapid acceleration or deceleration.

The display-device-communication module 1601C.4 includes instructions that, when executed, or circuits that, when activated, cause the computing device 1601 to communicate with the display device 1604, for example by sending one or more images or user interfaces to the display device 1604.

Furthermore, in some embodiments, the computing device 1601 also communicates with the light source 1603. For example, some embodiments of the computing device 1601 use the positional information from the position sensor 1607 to modulate the power of the light source 1603. In some embodiments, while the optical scope 1620 is undergoing extreme acceleration or deceleration, the computing device 1601 decreases the intensity of the light that is emitted by the light source 1603.

The scope of the claims is not limited to the above-described embodiments and includes various modifications and equivalent arrangements. Also, as used herein, the conjunction “or” generally refers to an inclusive “or,” although “or” may refer to an exclusive “or” if expressly indicated or if the context indicates that the “or” must be an exclusive “or.”

1. A system comprising:
a light source;
a spectrometer;
an optical scope that has a longitudinal axis and that includes a rotatable optical assembly, wherein the optical scope is coupled to the light source and to the spectrometer; and
an oscillating-drive device that is coupled to the optical scope, wherein the oscillating-drive device and the optical scope are configured such that the oscillating-drive device can rotate the rotatable optical assembly of the optical scope through greater than 90° of rotation around the longitudinal axis of the optical scope.

2. The system of claim 1, wherein the oscillating-drive device can rotate the rotatable optical assembly of the optical scope through at least 180° of rotation.

3. The system of claim 1, wherein the oscillating-drive device can rotate the rotatable optical assembly of the optical scope through at least 360° of rotation.

4. The system of claim 1, further comprising:
a spooling mechanism,
wherein the rotatable optical assembly includes one or more light-guiding components, and
wherein the spooling mechanism is configured to wind or unwind the one or more light-guiding components as the oscillating-drive device rotates the rotatable optical assembly.

5. The system of claim 4, wherein the one or more light-guiding components extend from the light source to a distal end of the optical scope or extend from the distal end of the optical scope to the spectrometer.

6. The system of claim 4, wherein the spooling mechanism comprises a first spool and a spring.

7. The system of claim 6, wherein the spooling mechanism further comprises at least one of a swing arm, a pulley, and a second spool.

8. The system of claim 1, wherein the rotatable optical assembly includes one or more of the following: a lens, a spacer, a mirror, a grating, and a light-guiding component.

9. The system of claim 1, wherein the optical scope does not include a rotary junction.

10. The system of claim 1, further comprising a sensor adapted to sense a position of the rotatable optical assembly.

11. The system of claim 10, wherein an image obtained by the system is constructed by removing areas where the rotatable optical assembly is rapidly accelerating or decelerating.

12. A system comprising:
an optical scope that includes a rotatable optical assembly and that has a longitudinal axis; and
an oscillating-drive device that is coupled to the optical scope, wherein the oscillating-drive device and the optical scope are configured such that the oscillating-drive device can oscillate the rotatable optical assembly through more than 90° of rotation around the longitudinal axis of the optical scope.

13. The system of claim 12, wherein the oscillating-drive device can oscillate the rotatable optical assembly of the optical scope through more than 180° of rotation.

14. The system of claim 12, wherein the oscillating-drive device can oscillate the rotatable optical assembly of the optical scope through more than 360° of rotation.

15. The system of claim 12, further comprising:
a spooling mechanism,
wherein the rotatable optical assembly includes one or more light-guiding components, and
wherein the spooling mechanism is configured to wind or unwind the one or more light-guiding components when the oscillating-drive device rotates the rotatable optical assembly.

16. The system of claim 15, wherein the spooling mechanism comprises a first spool.

17. The system of claim 16, wherein the spooling mechanism further comprises at least one of a second spool, a swing arm, and a pulley.

18. The system of claim 12, wherein the rotatable optical assembly includes one or more of the following: a lens, a spacer, a mirror, a grating, and a light-guiding component.

19. The system of claim 12, further comprising a sensor adapted to sense a rotational position of the rotatable optical assembly.

20. A system comprising:
an optical scope that includes a rotatable optical assembly, wherein the rotatable optical assembly includes one or more light-guiding components; and
a spooling mechanism, wherein the spooling mechanism is configured to wind or unwind the one or more light-guiding components when the rotatable optical assembly rotates.

21. The system of claim 20, wherein the spooling mechanism comprises at least one of a spool, a spring, a swing arm, and a pulley.
